(19) United States
(12) Patent Application Publication
(10) Pub. No.: US 2024/0277627 A1
(43) Pub. Date: Aug. 22, 2024

(54) NOVEL FLUOROCHEMICAL CARDIAC RESUSCITATION

(71) Applicant: NUVOX PHARMA LLC, Tucson, AZ (US)

(72) Inventor: Evan C. UNGER, Tucson, AZ (US)

(21) Appl. No.: 18/460,334

(22) Filed: Sep. 1, 2023

Related U.S. Application Data
(60) Provisional application No. 63/486,324, filed on Feb. 22, 2023.

(51) Int. Cl. A61K 31/02; A61K 9/107; A61K 47/10; A61K 47/24; A61K 47/26

(52) U.S. Cl. CPC A61K 31/02; A61K 9/1075; A61K 47/10; A61K 47/24; A61K 47/26

(57) ABSTRACT

The invention provides pharmaceutical compositions and dosage forms of fluorocarbon emulsions that are useful for treating blocked blood flow, such as from cardiac arrest, as well as methods of preparation and use thereof.

### TECHNICAL FIELDS OF THE INVENTION

This invention relates to pharmaceutical compositions and methods of their preparation and therapeutic use. More particularly, the invention relates to pharmaceutical compositions and dosage forms of fluorocarbon emulsions that are useful in treating cardiac arrest and related diseases and conditions, as well as methods of preparation and use thereof.

### BACKGROUND OF THE INVENTION

The American Heart Association's report Heart and Stroke Statistics—2022 Update (https://www.ahajournals.org/doi/10.1161/CIR.0000000000001052shows that cardiac arrest remains a public health crisis. There are more than 356,000 out-of-hospital cardiac arrests (OHCA) annually in the U.S., nearly 90% of them fatal. The incidence of EMS-assessed non-traumatic OHCA in people of any age is estimated to be 356,461, or nearly 1,000 people each day. Survival to hospital discharge after EMS-treated cardiac arrest languishes at about 10%.

The treatment of cardiac arrest involves chest compressions, ventilation, and early defibrillation. Adrenaline 1:10,000 10 microgram/kg=0.1 mL/kg is administered for shockable and non-shockable cardiac arrest and non vagus-induced bradycardia (intravenous, i.v., or intramuscular, i.m.). Amiodarone is administered for shock resistant ventricular fibrillation or pulseless ventricular tachycardia. Atropine, lidocaine, sodium bicarbonate and calcium are also administered in specific situations.

### SUMMARY OF THE INVENTION

The invention is based in part on the unexpected discovery of pharmaceutical compositions of certain fluorocarbons that exhibit therapeutic properties and can be safely and reliably used for treating cardiac arrest. While in the past, PFCs have been evaluated for cardiac arrest, this has only been tested with pulmonary administration and using PFCs that require high doses for efficacy. Dodecafluoropentane emulsion (DDFPe) is active at less than 1<100th the dose in terms of gram weight of higher molecular weight PFCs and can be provided at doses which are effective to deliver oxygen to hypoxic tissue, for example, cardiac tissue which is hypoxic due to ischemia and/or cardiac arrest.

In particular, fluorocarbon materials with boiling points between about −34° C. to about 105° C., for example, dodecafluoropentane emulsion (DDFPe), have been discovered to improve survival and outcomes in patients with cardiac arrest, particularly when blood flow has stopped. The unique pharmaceutical compositions and methods of use disclosed herein enable timely and substantial restoration of critical oxygen supply to affected organs, thus leading to reduced organ and tissue damages and improved treatment outcome. This is unexpected as a related perfluorocarbon oxygen formulation, Oxygent (perflubron), does not resuscitate from cardiac arrest/blocked blood flow. Oxygent is comprised of perfluorooctyl bromide with a boiling point of 142° C.

In one aspect, the invention generally relates to a method for treating cardiac arrest, or a related disease in which blood flow has stopped or condition thereof, comprising administering to a subject in need thereof a pharmaceutical composition comprising a therapeutically effective dosage of a fluorocarbon having a boiling point between about −36° C. to about 105° C., and a pharmaceutically acceptable carrier or excipient.

In another aspect, the invention generally relates to a unit dosage form of a pharmaceutical composition in the form of an emulsion comprising a dosage of a fluorocarbon having a boiling point between about −36° C. and about +105° C. therapeutically effective to treat cardiac arrest, in a mammal, including a human, and a pharmaceutically acceptable carrier or excipient.

### Definitions

Unless defined otherwise, all technical and scientific terms used herein have the same meaning as commonly understood by one of ordinary skill in the art to which this invention belongs.

As used herein, the term “emulsion” refers to a suspension or emulsion of nanodroplets or microbubbles in aqueous media. Nanodroplets refers to submicron droplets comprising a liquid fluorocarbon, e.g., ranging from 4 carbon to 8 carbons in length.

As used herein, “administration” of a disclosed compound or composition encompasses the delivery to a subject of a pharmaceutical composition using any suitable formulation or route of administration, as discussed herein.

As used herein, the terms “effective amount” or “therapeutically effective amount” refer to that amount of a compound or pharmaceutical composition described herein that is sufficient to effect the intended benefit including, but not limited to, disease treatment, as illustrated herein. The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. For instance, treatment herein may achieve an increase in a subject's oxygen saturation level or an improvement or restoration of oxygen supply. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the term “therapeutic effect” refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

### DETAILED DESCRIPTION OF THE INVENTION

The invention provides compositions of certain fluorocarbons that exhibit exceptional therapeutic properties and can be safely and reliably used for cardiac arrest. The unique pharmaceutical compositions and methods of use disclosed herein enable timely intervention and timely restoration of critical oxygen supply to affected organs when blood flow has stopped, thus leading to improved treatment outcome.

Different fluorocarbons have different boiling points and different capacity to deliver oxygen in a therapeutic setting. For example, dodecafluoropentane emulsion (DDFPe) has markedly different properties than prior PFCs that have been tested as oxygen therapeutics. DDFPe is a 2% w/vol emulsion of n-dodecafluoropentane (C₅F₁₂). DDFP has a boiling point of 29° C., less than body temperature. The DDFP in emulsion form may be stabilized by a surfactant, e.g. 0.3% w/vol PEG-Telomer-B fluorosurfactant. DDFPe particles are about 200 nm in diameter, about 1/15^{th} the diameter of a red blood cell, and is effective to deliver oxygen to hypoxic tissue at doses of 0.1 to 1.0 cc/kg-0.002 to 0.02 grams of PFC per kg body weight. Furthermore, DDFP is not metabolized and clears from the body via exhalation, with a terminal half-life in humans of about 90 minutes. PFCs having a much higher boiling point are only active with high FiO₂, require doses greater than 1 gram per kg body weight, are viscous and difficult to administer, and are not rapidly eliminated from the body.

DDFPe was originally developed as an ultrasound contrast agent. For this application it was necessary to create microbubbles and this was accomplished by creating negative pressure in the syringe prior to IV administration (hypobaric activation). It was approved in Europe by the EMA and approvable by the US FDA. Because other ultrasound contrast agents were approved and the market was small, the company developing DDFPe halted development of it as an ultrasound contrast agent. It was subsequently discovered that DDFPe delivered>100× as much oxygen per gram PFC than the high-boiling point liquid PFCs that had previously been tested as oxygen therapeutics. For oxygen delivery applications, in some embodiments, a neat emulsion is administered, e.g., by simple IV injection without creating microbubbles at the point of injection, e.g. without hypobaric activation as described above.

In one aspect, the invention generally relates to a method for treating cardiac arrest, comprising administering to a subject in need thereof a pharmaceutical composition comprising a therapeutically effective dosage of a fluorocarbon having a boiling point between about −36° C. to about 105° C., and a pharmaceutically acceptable carrier or excipient. In certain embodiments, the fluorocarbon has a boiling point higher than room temperature and lower than body temperature, e.g. a boiling point greater than about 20° C. and less than about 37° C., e.g., between about 22° C. and about 35° C.

Boiling point for purposes of this disclosure is measured at 1 atm of pressure; however, in certain embodiments, the fluorocarbon may be packaged and delivered at elevated pressures to facilitate dissolution of the fluorocarbon or at reduced pressures to facilitate release of the fluorocarbon.

In certain embodiments, the fluorocarbon is preferably stabilized in the form of an emulsion. In certain preferred embodiments, the pharmaceutical composition is an emulsion, e.g., a homogenized emulsion.

Depending on the fluorocarbon used, the fluorocarbon may be present as nanodroplets or in microbubbles in the emulsion.

In certain embodiments, the emulsions comprise particles having sizes in the range from about 0.5 μm to about 5 μm (e.g., about 0.5 μm to about 2 μm). In certain embodiments, the emulsions comprise particles (nanodroplets or microbubbles) having sizes less than about 1 μm in size. In certain embodiments, the emulsions comprise particles having sizes less than about 500 nm. In certain embodiments, the emulsions comprise particles having sizes less than about 250 nm.

In certain embodiments, the boiling point of the fluorocarbon used is preferably between about −4° C. and about 105° C. In certain embodiments, the boiling point of the fluorocarbon used is preferably between about −4° C. and about 60° C. In certain embodiments, the boiling point of the fluorocarbon used is preferably between about 28° C. and about 60° C.

In certain embodiments, the fluorocarbon used preferably has between 4 and 8 linear and/or branched carbon atoms with from about 10 to about 18 fluorine atoms.

Fluorocarbons useful in the invention include perfluorobutane, perfluoropentane, perfluorohexane, perfluoroheptane and perfluorooctane, or a mixture of two of more thereof. In certain embodiments, the pharmaceutical composition utilizes perfluorohexane and/or perfluoropentane. In certain embodiments, the pharmaceutical composition utilizes perfluoropentane. Perfluoropentane may comprise isomers of dodecafluoro-n-pentane (dodecafluoropentane) and dodecafluoro-iso-pentane. In certain embodiments, the fluorocarbon is dodecafluoropentane.

The fluorocarbon accounts for a concentration in the emulsion from about 0.1% w/vol to about 80% w/vol. In certain embodiments, the fluorocarbon accounts for a concentration in the emulsion from about 1% w/vol to about 20% w/vol.

In certain embodiments, the emulsion has from about 0.5 to about 20% w/vol of fluorocarbon. In certain embodiments, the emulsion has between about 1 and about 10% w/vol fluorocarbon. In certain embodiments, the emulsion has between about 1 and about 5% w/vol fluorocarbon. In certain embodiments, the emulsion has between about 3 and about 7% w/vol fluorocarbon. In certain embodiments, the emulsion has from about 0.5 to about 2% w/vol of fluorocarbon. In certain embodiments, the emulsion has between about 1 and about 3% w/vol fluorocarbon. In certain embodiments, the emulsion has between about 2 and about 4% w/vol fluorocarbon. In certain embodiments, the emulsion has between about 3 and about 5% w/vol fluorocarbon.

In certain embodiments, the pharmaceutical composition comprises one or more phospholipids having carbon chains ranging from about 12 carbons to about 18 carbons in length.

In certain embodiments, the phospholipids accounts for a weight percent in the pharmaceutical composition from about 1.5% to about 10%.

In certain embodiments, the fluorocarbon is stabilized by one or more surfactants. For example, surfactants may be one or more fluorosurfactants such as PEG-Telomer-B, CAPSTONE, diacylglycerophospholipids, cholesterol, and/or other surfactants known in the art. In certain embodiments, the surfactant(s) utilized comprise one or more fluorosurfactants and one or more phospholipids.

In certain embodiments the fluorocarbon is stabilized by a PEG-Telomer-B fluorosurfactant, which is a custom purified perfluorocarbon-oligoethyleneoxyalcohol surfactant that is obtained starting with Dupont Zonyl FSO-100 or Dupont Zonyl FSO. The purified product contains a mixture of F4, F6, F8, F10, F12, F14 and F16 compounds in the approximate ranges of relative amounts. F4≤0.3%, F6 53-69%, F8 24-36%, F10 5-11%, F12, F14 and F16 combined ≤1.6%. Very small amounts of perfluoroethyl (F2) congener may be present in the product. Here, F refers to the number of perfluorinated carbons in the perfluorocarbon moiety present. The oligoethyleneoxyalcohol function is a distribution of oligoethylene alcohol functions (CH₂CH₂O)ₙ where n=2, 3, 4, 5, 6, 7, 8, 9, 10, 11, 12, 13, 14, 15, or 16 are present. Very small amounts of n>16 may be present in the product.

In certain embodiments, the surfactant(s) is incorporated into the emulsion in amounts ranging from about 0.1% weight volume to about 10% weight volume. In certain embodiments, the surfactant(s) is incorporated into the emulsion in amounts ranging from about 0.2% w/vol to about 2% w/vol.

In certain embodiments, the pharmaceutical composition is adapted for intravenous administration. For example, in certain embodiments, the pharmaceutical composition is buffered, e.g., using phosphate-buffered saline to approximately neutral pH. In certain embodiments, the pharmaceutical composition is osmotically adjusted, e.g., using an isotonic saline solution (e.g., 0.9% saline) and optionally further osmotically adjusted using a pharmaceutically acceptable sugar or sugar alcohol, for example sucrose or maltilol. For example, the pharmaceutical composition may be an emulsion for intravenous administration comprising a fluorocarbon (e.g., having a boiling point between about −36° C. to about 105° C.), surfactant, buffer, saline, and optionally a pharmaceutically acceptable sugar, sugar alcohol, or mixture thereof. For example, in one embodiment, the pharmaceutical composition is an emulsion for intravenous administration comprising 2% w/vol DDFP with 0.3% w/vol PEG-Telomer B fluorosurfactant, saline solution (e.g., 0.9% saline), 30% w/vol sucrose, and phosphate buffered saline, at approximately neutral pH.

In certain embodiments, the pharmaceutical composition is administered intravenously when blood flow has stopped or within four hours of blood flow interruption and preferably within two hours of blood flow interruption.

In certain embodiments, the pharmaceutical composition may be administered as an IV bolus. In certain embodiments, the pharmaceutical composition may be administered by sustained IV infusion. In certain embodiments, the pharmaceutical composition is injected intravenously via bolus or slow IV push over about 3 to about 5 minutes at doses ranging from about 0.2 mg/kg to about 20 mg/kg (e.g., about 0.2 mg/kg to about 10 mg/kg, about 0.2 mg/kg to about 5 mg/kg, about 0.2 mg/kg to about 2 mg/kg, about 1 mg/kg to about 20 mg/kg, about 5 mg/kg to about 20 mg/kg, about 10 mg/kg to about 20 mg/kg).

The volume of FC administered will vary depending upon the concentration of FC in the final product but for a 70-100 kg human subject might vary from about 1 ml to about 200 ml. In the example shown below the dose was 0.5 mL/kg or 20 cc in a 20 kg pig. With allometric scaling the predicted dose would be about 0.35 mL/kg in a human subject.

In certain embodiments, the pharmaceutical composition is preferably injected intravenously as a sustained IV infusion at a rate of from about 0.5 mg/kg/hour up to about 7.0 mg/kg/hour (e.g., about 0.5 mg/kg/hour up to about 5.0 mg/kg/hour, about 0.5 mg/kg/hour up to about 3.0 mg/kg/hour, about 0.5 mg/kg/hour up to about 2.0 mg/kg/hour, about 1 mg/kg/hour up to about 7.0 mg/kg/hour, about 2 mg/kg/hour up to about 7.0 mg/kg/hour, about 3 mg/kg/hour up to about 7.0 mg/kg/hour).

The concentration of fluorocarbon in the emulsion can be increased, for example, up to about 60% weight/vol if desired, to minimize the volume injected.

In certain embodiments, the pharmaceutical composition is administered as an IV infusion at a rate of from about 0.2 mg/kg to about 40 mg/kg per hour (e.g., about 0.2 mg/kg to about 20 mg/kg per hour, about 0.2 mg/kg to about 10 mg/kg per hour, about 0.2 mg/kg to about 5 mg/kg per hour, about 0.2 mg/kg to about 2 mg/kg per hour, about 1 mg/kg to about 40 mg/kg per hour, about 5 mg/kg to about 40 mg/kg per hour, about 10 mg/kg to about 40 mg/kg per hour, about 1 mg/kg to about 10 mg/kg per hour, about 5 mg/kg to about 20 mg/kg per hour).

In certain embodiments, a dose of the pharmaceutical composition is repeated as needed, for example from 1 to about 50 times (e.g., about from 1 to about 25 times, from 1 to about 10 times, from 1 to about 5 times, from 1 to about 3 times, from 2 to about 10 times).

In certain embodiments, the method comprises administering the subject a second therapeutic agent, before, during or after the administration of the fluorocarbon containing pharmaceutical composition. The fluorocarbon emulsions of the invention may be co-administered with one or more other suitable second agents, or one or more such second agents may be incorporated into the fluorocarbon emulsion.

Exemplary second therapeutic agents include, but not limited to adrenaline, atropine, amiodarone, lidocaine, sodium bicarbonate, and calcium.

The compositions and methods of the invention are useful in cardiac arrest or condition thereof where blood flow has stopped or been interrupted.

In another aspect, the invention generally relates to a unit dosage form of a pharmaceutical composition in the form of an emulsion comprising a dosage of a fluorocarbon having a boiling point between about −36° C. and about +105° C. therapeutically effective to treat cardiac arrest, or a related disease or disorder thereof where blood flow has stopped or been interrupted, in a mammal, including a human, and a pharmaceutically acceptable carrier or excipient.

As disclosed herein, fluorocarbons useful in the invention include perfluoropropane, perfluorobutane, perfluoropentane, perfluorohexane, perfluoroheptane and perfluorooctane, or a mixture of two of more thereof. In certain embodiments, the pharmaceutical composition utilizes perfluorohexane and/or perfluoropentane. In certain embodiments, the pharmaceutical composition utilizes perfluoropentane. Perfluoropentane may comprise isomers of dodecafluoro-n-pentane (dodecafluoropentane) and dodecafluoro-iso-pentane. In certain embodiments, the fluorocarbon is dodecafluoropentane. In certain embodiments of the unit dosage form, the fluorocarbon is dodecafluoropentane.

Any suitable therapeutically effective unit dosage form may be employed, for example, comprising about 1% to about 10% w/vol of the fluorocarbon. In certain embodiments, the unit dosage form comprises about 2% to about 7% w/vol of the fluorocarbon. In certain embodiments, the unit dosage form comprises from about 1 mg/kg body weight to about 7 mg/kg (e.g., about 1 mg/kg body weight to about 5 mg/kg, about 1 mg/kg body weight to about 3 mg/kg, about 2 mg/kg body weight to about 7 mg/kg, about 3 mg/kg body weight to about 7 mg/kg) body weight of fluorocarbon.

Antioxidants may also be used in the invention to improve the activity of the fluorocarbon. Examples of useful antioxidants include n-acetylcysteine, ascorbic acid, and α-tocopherol. In certain embodiments, n-acetylcysteine can be administered at 150 mg/Kg for 30 min then 20 mg/Kg/h plus bolus doses of 1 g ascorbic acid and 400 mg α-tocopherol.

The following examples are presented to further illustrate to persons skilled in the art how to make and use the invention. These examples are not intended as a limitation, however, upon the scope of the invention.

### EXAMPLES

Table 1 below shows fluorocarbons useful in the invention. In general, fully fluorinated fluorocarbons ranging in molecular weight from about 188 to about 438 grams/mole are useful in the invention. The fluorocarbons may be linear or branched or cyclic but have boiling points ranging from about −37° C. to about 100° C. More preferably the boiling point of the fluorocarbon is close to the physiologic temperature in the human body, or slightly less than the temperature in the human body or that of a mammal. Most preferred is the fluorocarbon perfluoropentane which may exist as n-perfluoropentane, the linear isomer, or the branched isomers of iso-perfluoropentane or neo-perfluoropentane.

Most preferably the fluorocarbon is stabilized in an emulsion. Preferably the emulsified suspension of nanodroplets of fluorocarbon represents a liquid in liquid suspension, although with the lower boiling point fluorocarbons such as perfluoropropane or perfluorobutane the product may represent a suspension of microbubbles of fluorocarbon in liquid. Most preferably, however, the product comprises a liquid in liquid suspension. Preferably the size of the nanodroplets in the liquid in liquid suspension ranges from about 1 nm to about 1,000 nm and more preferably from about 50 to about 500 nm and still more preferably from about 100 nm to about 250 nm. In the case of a microbubble formulation the mean size of the microbubbles ranges from about 0.2 to about 5 microns and more preferably around 1 Micron.

TABLE 1
Fluorocarbons Useful in the Invention

| | Molecular Weight | | |
|---|---|---|---|
| Chemical Entity | (g/mol) | Boiling Point | |
| Perfluoropropane | 188.02 | −36.7° | C. |
| Perfluorobutane | 238.03 | −1.7° | C. |
| n-Perfluoropentane | 288.04 | 28° | C. |
| Iso-Perfluoropentane | 288.04 | 28-30° | C. |
| Perfluorohexane | 338.04 | 56° | C. |
| Perfluoroheptane | 388.05 | 56° | C. |
| Perfluoro-octane | 438.06 | 103-104° | C. |

Generally, one or more surfactants are used to stabilize the emulsion. Useful surfactants include fluorosurfactants, e.g., PEG-telomer-B (PTB) and phospholipids. A preferred product in the invention is DDFPe. A typical DDFPe may comprise 2% w/vol DDFP with 0.3% w/vol PTB in a saline solution with 30% w/vol sucrose with phosphate buffered saline at near neutral pH also referred to as NanO₂™. By adjusting the concentrations of DDFP and surfactant, however, formulations may be prepared ranging from about 0.1% w/vol DDFP up to 100% w/vol DDFP. More preferably the concentration of the DDFP ranges from about 1% w/vol DDFP to about 50% w/vol DDFP and more preferably from about 1% w/vol DDFP to about 10% w/vol DDFP. Most preferred is the 2% w/vol DDFP concentration in NanO₂. NanO₂ comprises a mixture of the isomers of 99% n-perfluoropentane and 1% iso-perfluoropentane but the invention may comprise one or more of the n-, iso- or neo-perfluoropentane isomers.

The volumes administered to treat cardiac arrest will vary depending upon the concentration of DDFP. The example below formulation comprises 2% w/vol DDFP with 0.3% w/vol PEG-Telomer-B in the formulation. The preferred route of administration of the fluorocarbon is intravenous (IV), via bolus, slow IV push or sustained infusion. Other routes of administration such as inhalation, intraperitoneal and oral may also be practiced.

### Experiments

### Example 1

Cardiac arrest was produced in pigs by placing spinal needles percutaneously near the heart and applying an electrical current via three 9V batteries in series, leading to ventricular fibrillation. Absence of blood flow was confirmed on arterial line for 5 min before starting CPR. After 2 min of CPR, NanO₂™ infusion was commenced at a fixed dose of 20 mL over 10 minutes (40 lb. pigs, dose of NanO2=0.5 mL/kg). Standard ACLS resuscitation was followed otherwise, with defibrillation, epinephrine, amiodarone as needed.

Investigators at the University of Florida, led by Torben Becker, MD, PhD, Associate Professor, Chief-Division of Critical Care Medicine, completed 12 experiments, of which 7 animals were given NanO₂ and 5 animals received placebo. The study was funded by the Zoll Foundation.

Out of 5 placebo animals, 2 (40%) achieved return of spontaneous circulation (ROSC) and 1 (20%) survived to 96 hrs. Out of 7 NanO₂ treated animals, 6 (85%) achieved ROSC and 4 (57%) survived to 96 hrs.

In other words, the NanO₂ group had double the ROSC and survival rates of the placebo group.

### Example 2

The above was repeated except that a dose of Oxygent (60% w/vol perflubron) was administered instead of NanO₂. Oxygent is an emulsion of 60% w/vol perfluorooctylbromide which has a boiling point of 142° C. The animal was not resuscitated from administration of Oxygent.

While the preferred embodiments of the present invention have been illustrated in detail, it should be apparent that modifications and adaptations to those embodiments may occur to one skilled in the art without departing from the scope of the present invention.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the following description, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A method for treating cardiac arrest, or a related disease or condition thereof, comprising administering to a subject in which blood flow has stopped, a pharmaceutical composition comprising a therapeutically effective dosage of a fluorocarbon having a boiling point between about −36° C. to about 105° C., and a pharmaceutically acceptable carrier or excipient.

2. The method of claim 1, wherein the pharmaceutical composition is an emulsion.

3. The method of claim 1, wherein the fluorocarbon is provided as nanodroplets or in microbubbles.

4. The method of claim 3, wherein the emulsion comprises nanodroplets or microbubbles less than 1 micron in size.

5. The method of claim 3, wherein the nanodroplets or in microbubbles have size from about 0.5 microns to about 5 microns.

6. The method of claim 1, wherein the fluorocarbon comprises perfluoropropane, perfluorobutane, perfluoropentane, perfluorohexane, perfluoroheptane, perfluorooctane, or a mixture of two of more thereof.

7. The method of claim 6, wherein the fluorocarbon comprises dodecafluoropentane.

8. The method of claim 1, wherein the fluorocarbon is present at a concentration ranging from about 1% w/vol to about 20% w/vol.

9. The method of claim 1, wherein the fluorocarbon composition comprises a surfactant.

10. The method of claim 9, wherein the surfactant is PEG-Telomere-B fluorosurfactant.

11. The method of claim 1, wherein the pharmaceutical composition comprises one or more phospholipids having carbon chains ranging from about 12 carbons to about 18 carbons in length.

12. The method of claim 1, wherein the pharmaceutical composition is administered intravenously.

13. The method of claim 12, wherein the pharmaceutical composition is injected intravenously via bolus or slow IV push over about 3 to about 5 minutes at doses ranging from about 0.2 mg/kg to about 20 mg/kg.

14. The method of claim 12, wherein the pharmaceutical composition is administered as an IV infusion at a rate of from about 0.2 mg/kg to about 40 mg/kg per hour.

15. The method of claim 1, wherein the pharmaceutical composition is an emulsion for intravenous administration comprising the fluorocarbon; a surfactant; saline; buffer; and optionally a sugar, sugar alcohol, or mixture thereof.

16. The method of claim 1, wherein the fluorocarbon has a boiling point greater than about 20° C. and less than about 37° C.

17. The method of claim 1,
wherein the fluorocarbon is dodecafluoropentane (DDFP);
wherein the pharmaceutical composition is in the form of an emulsion comprising about 2% w/vol of DDFP and about 0.3% w/vol PEG-Telomer-B fluorosurfactant in a saline solution with about 30% w/vol sucrose with phosphate buffered saline at near neutral pH, and wherein the emulsion particles are about 200 nm in diameter;
wherein the pharmaceutical composition is administered intravenously by IV infusion; and
wherein the therapeutically effective dosage is about 2 mg to about 20 mg of DDFP per kg body weight.
